(19) United States
(12) Patent Application Publication
(10) Pub. No.: US 2004/0167631 A1
(43) Pub. Date: Aug. 26, 2004

(54) Fixation surface for ankle prosthesis

(76) Inventors: Kenny Luchesi, Austin, TX (US); Steven Brown, Pflugerville, TX (US); Mengke Zhu, Austin, TX (US)

Correspondence Address: ZIMMER TECHNOLOGY, INC., 150 N. WACKER DRIVE, SUITE 1200, CHICAGO, IL, 60606, US

(21) Appl. No.: 10/372,367

(22) Filed: Feb. 21, 2003

(51) Int. Cl. A61F 2/42

(52) U.S. Cl. 623/21.18

(57) ABSTRACT

An ankle prosthesis comprising two separate components, a tibial component and a talar component. Each component has an articulating surface and a fixation surface. The fixation surfaces are cement retained and include an elongated keel and a plurality of cement grooves to bond with bone cement and bone.

### FIELD OF THE INVENTION

The disclosure herein generally relates to implantable orthopedic prostheses and, more particularly, to fixation surfaces for cement retained ankle prostheses.

### BACKGROUND OF THE INVENTION

In the United States alone, thousands of ankle arthrodesis and ankle arthroplasty procedures are performed each year. Osteoarthritis arthritis, rheumatoid arthritis, and post-traumatic arthritis are the most common reasons for these procedures. In some severe arthritic cases, cartilage and synovium surrounding the ankle wear down so underlying bones grind directly on each other. Arthritis of the ankle, in all these forms, can cause severe pain and disability for the patient.

Patients have several surgical treatment options for arthritis of the ankle. These options include ankle arthroscopy, ankle arthrotomy and debridement (opening the joint and removing bone spurs), osteotomy (cutting bone to realign the joint), ankle fusion or arthrodesis (removing the joint and making it stiff), and total ankle arthroplasty (replacing the joint with an ankle prosthesis).

To date, ankle arthrodesis is one widely accepted procedure for treating arthritis in the ankle. This procedure though, has many shortcomings, such as infection, skin slough, nerve injury or entrapment, and nonunion or mal-union. Further yet, a big disadvantage with this procedure is that the patient must sacrifice ankle motion and mobility to relieve pain. This sacrifice, in turn, can increase stress on other joints. Some surgeons, for instance, believe that arthrodesis compromises the knee and hip joints because the body attempts to compensate for the lost range of motion at the ankle. Given these shortcomings, more focus has been devoted to ankle arthroplasty as an alternative to ankle arthrodesis.

In ankle arthroplasty, portions of the natural ankle joint are replaced with prosthetic components. These components include a tibial component implanted into the end of the tibia and a talar component implanted into the end of the talus. These two components include articulation surfaces that articulate against each other in an attempt to simulate the natural articulating motion between the tibia and talus. The components are made of materials that exhibit a low coefficient of friction when they articulate during normal range of motion.

Much effort has been devoted to designing ankle prostheses that restore normal, pain-free functions to the ankle joint. To date, these prostheses have had limited success.

The first generation of ankle prosthesis was not designed to emulate the biomechanical characteristics of the natural joint. The designs required excessive bone resection and ultimately had a high rate of long term failure.

A second generation of ankle prosthesis improved on the first generation but still exhibited numerous disadvantages. Many of the designs have lead to complications and failures. In short, these prostheses have failed to emulate the natural kinematics of the ankle joint.

It therefore would be advantageous to provide a new generation of ankle prostheses that have significant advantages over the first and second generation of prostheses.

### SUMMARY OF THE INVENTION

The present invention is directed toward ankle prostheses generally comprising two separate components, a tibial component and a talar component. Each component has an articulating surface and a fixation surface oppositely disposed from the articulating surface. The fixation surface is adapted to engage and fix to bone and includes a keel and a plurality of cement retaining grooves. The keel may have different shapes that are adapted to strongly adhere to bone cement. These shapes include, but are not limited to, straight rectangular shape, a dovetail shape, an I-beam shape, and a T-shape. Further, the cement grooves may have different shapes that are adapted to strongly adhere to bone cement. These shapes include, but are not limited to, circular, spherical, and dovetail. The keel and cement grooves can be combined to form the fixation surface.

One important advantage of the present invention is that the fixation surfaces use keels and/or cement retaining grooves. The keels and grooves have shapes that are adapted to strongly bond with cement and firmly retain the tibial and talar components to bone.

Another advantage of the present invention is that, in some embodiments, the fixation surfaces simultaneously use both cement grooves and a keel. The combination of both of these features provides excellent bonding and strength between the fixation surface and bone when cement is applied. The use of keels and cement grooves are particularly important in ankle prostheses since the loads generated in the ankle joint are extremely high. The prosthesis must be strongly affixed to bone to withstand these loads and not move, loosen, or otherwise fail.

Another aspect of the present invention is that the fixation surfaces of the present invention can be utilized with various designs of prosthetic ankles. The fixation surface, for example, can be used with an ankle prosthesis that uses sphere-on-sphere articulating surfaces. This interface more closely emulates the natural kinematics of the ankle joint and results in numerous advantages over prior designs.

As one advantage, the sphere-on-sphere design decreases torsional forces across the ankle joint. These forces can result from a slight mal-alignment in any plane or high physiological stresses across the ankle joint.

As another advantage, the spherical design increases or maximizes the surface area of contact between the tibial and talar prosthetic component. As such, weight bearing loads are more evenly distributed across a larger area. This distribution is extremely important at the ankle joint. Here, loads are often eccentric (i.e., not central and evenly distributed). An uneven distribution of force causes compression on one side of the bone and a lift-off force on the opposite side. Expanding the surface area of articulating surfaces decreases the overall force per unit area. Further yet, the increased spherical area decreases angular stress or shearing that can cause micromotion. The spherical articulating surfaces are thus more resistant to the negative affects of eccentric forces, shear stresses, and micromotion.

As yet another advantage, one embodiment uses metal-on-metal articulating surfaces. These components can be manufactured to have a thin or minimal thickness yet exhibit high strength. The thin components result in nominal resection of bone, leaving more natural bone on the talus and tibia. As a result, the fixation between the bone engaging surfaces of the implant and cortical bone is strong. The added strength in this fixation prevents or reduces subsidence and ultimately early failure of the prosthesis. In an extreme case where failure still occurs at a later date, sufficient bone will remain for other operative procedures, such as a revision surgery or fusion. This additional strength also makes the prosthesis less prone to fail mechanically. A strong prosthesis is particularly important at this joint since the forefoot acts as lever to magnify compressive forces between the tibial and talus. These forces, for example, can be 5-7 times the weight of the patient.

As yet another advantage, the, spherical design provides strong bone support. Good support at the ankle joint is fundamental for success of the prosthesis. Poor support often leads to subsidence, loosening, and ultimately failure. This support is notably critical at the talus since it is anatomically small and provides a limited area for fixation.

As still yet another advantage, another embodiment of the present invention uses metal-on-polymeric articulating surfaces. Here, the talar component is made of polyethylene and the tibial component is made of metal. The metallic talar component has the same advantages as the talar component in the metal-on-metal embodiment, such as high strength and reduced bone resection. The polymeric component can be formed of a highly cross-linked UHMWPe that exhibits high strength and extremely low friction and wear properties.

Other advantages and benefits of the present invention will be apparent from the accompanying drawings and detailed disclosure of the invention.

### BRIEF DESCRIPTION OF THE DRAWINGS

FIG. 1A is a top perspective view of a convex component of the ankle prosthesis.

FIG. 1B is a bottom perspective view of the convex component of FIG. 1A.

FIG. 2A is a, top perspective view of a concave component of the ankle prosthesis.

FIG. 2B is a bottom perspective view of the concave component of FIG. 2A.

FIG. 3 is a side view of the concave component attached to a tibia and the convex component attached to a talus.

FIG. 4 is a side view of the convex component attached to the tibia and the concave component attached to the talus.

FIG. 5 is a side view of the convex component with a straight keel on the fixation surface.

FIG. 6 is a side view of the convex component with a dovetail keel on the fixation surface.

FIG. 7 is a bottom perspective view of the convex component with an I-Beam keel on the fixation surface.

FIG. 8 is a bottom perspective view of another embodiment of the convex component with an I-Beam keel on the fixation surface.

FIG. 9 is a side view of a convex component with a keel on the fixation surface.

FIG. 10 is a cross sectional view taken through the lines A-A of the convex component of FIG. 9.

FIG. 11 is an enlarged view of a spherical cement groove taken along circle B in FIG. 10.

FIG. 12 is a side view of another embodiment of a convex component with a keel on the fixation surface.

FIG. 13 is a cross sectional view taken through the lines A-A of the convex component of FIG. 12.

FIG. 14 is an enlarged view of a dovetail cement groove taken along circle B in FIG. 13.

FIG. 15 is a bottom perspective view of a convex component with cement grooves and a keel on the fixation surface.

FIG. 16 is a bottom perspective view of a concave component with cement grooves and a keel on the fixation surface.

FIG. 17 is an ankle prosthetic system having multiple sized concave and convex components that are connectable together.

### DETAILED DESCRIPTION

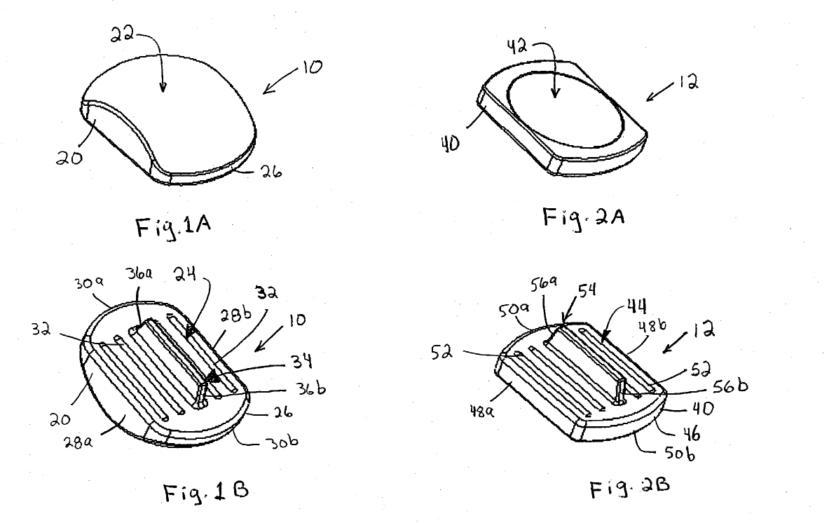

FIGS. 1A, 1B, 2A, and 2B together show the ankle prosthesis of the present invention. The prosthesis comprises two separate and distinct components, a convex component 10 and a concave component 12.

The convex component 10 has a body 20 with an articulating surface 22 on one side and a fixation surface 24 oppositely disposed on the other side. An outer wall 26 extends around a perimeter and includes two straight walls 28a and 28b and two rounded walls 30a and 30b. The perimeter forms a square or rectangular shape with rounded end walls. Articulating surface 22 has a smooth outer surface that has a spherical, convex shape. Preferably, surface 22 is symmetric with a continuous contour. Fixation surface 24 is flat or planar with a plurality of parallel cement grooves 32. These grooves extend substantially the entire length of the fixation surface. A rectangular keel 34 extends outwardly from the fixation surface. Keel 34 has tapering end walls 36a and 36b and is parallel with the grooves 32 and is disposed along a longitudinal center of the fixation surface.

The concave component 12 has a body 40 with an articulating surface 42 on one side and a fixation surface 44 oppositely disposed on the other side. An outer wall 46 extends around a perimeter and includes two straight walls 48a and 48b and two rounded walls 50a and 50b. The perimeter forms a square or rectangular shape with rounded end walls. Articulating surface 42 has a smooth outer surface that has a spherical, concave shape. Preferably, surface 42 is symmetric with a continuous contour. Fixation surface 44 is flat or planar with a plurality of parallel cement grooves 52. These grooves extend substantially the entire length of the fixation surface. A rectangular keel 54 extends outwardly from the fixation surface. Keel 54 has tapering end walls 56a and 56b and is parallel with the grooves 52 and is disposed along a longitudinal center of the fixation surface.

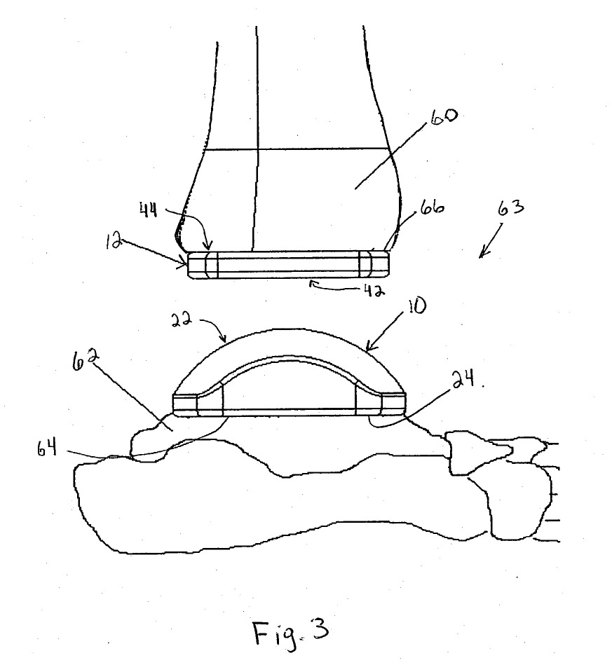

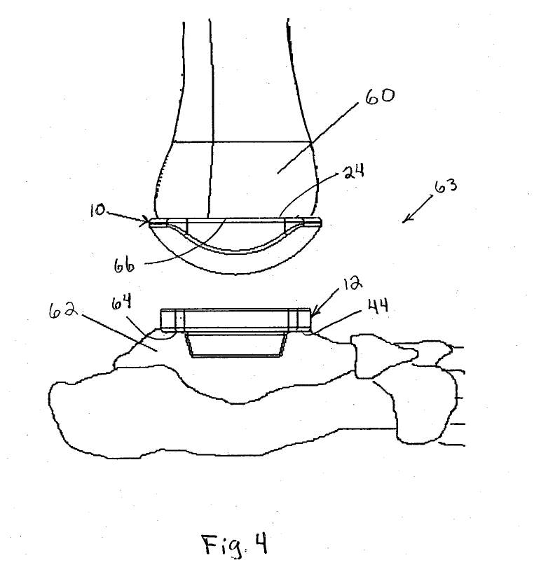

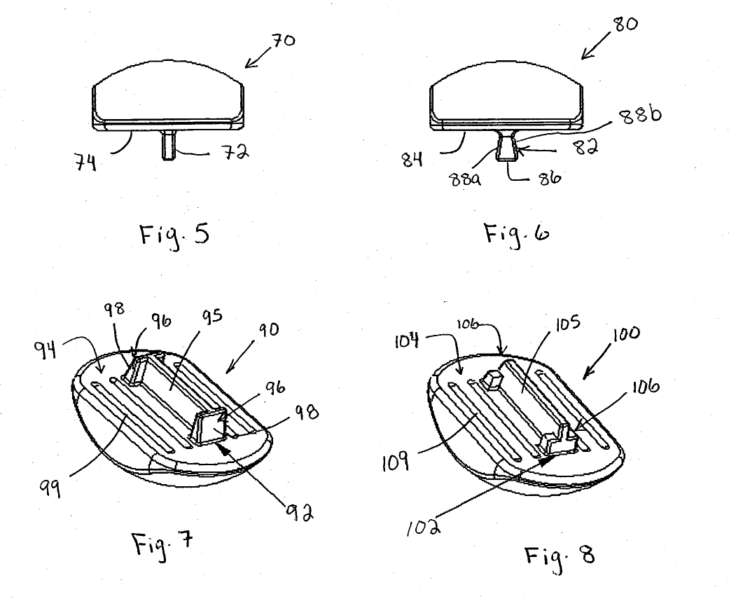

Looking to FIG. 3, concave component 12 is connected to the distal end of the tibia 60, and convex component 10 is connected to the talus 62. Together, these two components form the ankle prosthesis at the ankle joint 63 of a patient. More specifically, the fixation surface 24 of convex component 10 is engaged and connected to a planar surface 64 of talus 62. Likewise, fixation surface 44 of concave component 12 is engaged and connected to a planar surface 66 of tibia 60.

The articulating surfaces 22 and 42 are sized and shape to engage and slideably articulate with each other. During relative motion between the two surfaces, a portion of articulating surface 22 fits inside the concave cavity and smoothly rolls against articulating surface 42. During motion then, the two components articulate with each other with a sphere-on-sphere articulation. As noted, this spherical interface between the articulating surfaces is a critical component of the present invention.

As best shown in FIGS. 1A, 1B, 2A, 2B, and 3, the concave and convex components have substantially the same length and width. The components are thus shaped to articulate against each other in the naturally confined area of the ankle joint of a patient.

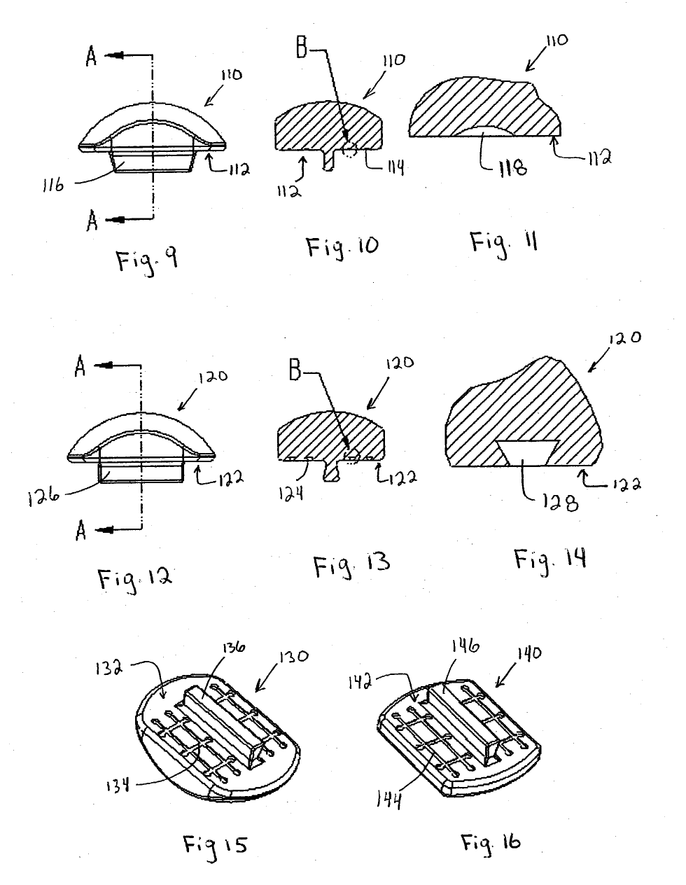

One advantage of the present invention is that the convex and concave components can be used on either the tibia or talus. In contrast to FIG. 3, the embodiment in FIG. 4 shows concave component 12 connected to the talus 62 and convex component 10 connected to the tibia 60. Together, these two components form the ankle prosthesis at the ankle joint 63 of a patient. More specifically, the fixation surface 24 of convex component 10 is engaged and connected to the planar surface 66 of tibia 60. Likewise, fixation surface 44 of concave component 12 is engaged and connected to the planar surface 64 of talus 62.

One important feature of the present invention is the combined use of both a keel and cement grooves on the same fixation surface. The combination of both of these features provides stronger bonding and retention between the fixation surface and cement than just a keel or just grooves.

Further, the specific shape of the grooves is important to the ability of the grooves to bond with cement and retain the prosthetic component fixed to the bone. The shapes discussed in the accompanying figures are particularly advantageous since they bond well with cement and provide a strong retention between the fixation surface and the cement.

Likewise, the specific shape of the keels is important to the ability of the keels to bond with cement and retain the prosthetic component fixed to the bone. The shapes discussed in the accompanying figures are particularly advantageous since they bond well with cement and provide a strong retention between the fixation surface and the cement.

FIG. 5 shows a convex component 70 similarly configured to the convex component 10 discussed in connection with FIGS. 1A and 1B. Component 70 includes a straight keel 72 that extends downwardly from fixation surface 74. The keel has an elongated rectangular shape and is centrally located on the fixation surface.

FIG. 6 shows a convex component 80 similarly configured to the convex component 10 discussed in connection with FIGS. 1A and 1B. Component 80 though includes a dovetail keel 82 that extends downwardly from fixation surface 84. The keel has an elongated dovetail shape with an end surface 86 and two sides 88a and 88b that taper inwardly toward the fixation surface 84.

The dovetail shape is particularly advantageous because the tapering sides 88a and 88b strongly secure the fixation surface into cement. Specifically, the dovetail shape becomes wedged or anchored into the cement.

FIG. 7 shows a convex component 90 similarly configured to the convex component 10 discussed in connection with FIGS. 1A and 1B. Component 90 though includes an I-beam keel 92 that extends downwardly from fixation surface 94. The keel has an elongated rectangular center portion 95 and two substantially square ends 96. These ends have end walls 98 that taper inwardly toward the center of the component. In a bottom view, the keel has an “I” shape. The fixation surface is also provided with a plurality of cement grooves 99. These grooves are spaced apart and parallel to the keel.

The I-beam keel is particularly advantageous because the shape securely bonds in cement and prohibits movement of the component. End walls 98, for example, bond with cement and prevent anterior-posterior movement, while the center portion 95 bonds with cement and prevents medial-lateral movement or rotational movement.

FIG. 8 shows a convex component 100 similarly configured to the convex component 10 discussed in connection with FIGS. 1A and 1B. Component 100 though includes a “T” shaped keel 102 that extends downwardly from fixation surface 104. The keel has an elongated rectangular center portion 105 and two T-shaped ends 106. The fixation surface is also provided with a plurality of cement grooves 109. These grooves are spaced apart and parallel to the keel.

The T-shaped keel is particularly advantageous because the shape securely bonds in cement and prohibits movement of the component. End walls 106, for example, bond with cement and prevent anterior-posterior movement, while the center portion 105 bonds with cement and prevents medial-lateral movement or rotational movement.

As shown in FIGS. 1B, 2B, 7, and 8, the fixation surfaces can have a plurality of, cement grooves and keels adapted to receive and engage bone cement for cementing the components to surrounding bone. These cement grooves and keels can have various configurations. Further, the grooves can be utilized with different sizes and shapes of keels. Some of these variations and combinations are shown in FIGS. 9-16.

FIGS. 9-11 show a convex component 110 having a fixation surface 112 with a plurality of cement grooves 114 and a rectangular straight keel 116. These grooves have a partial spherical shape or circular shape 118 in cross section (illustrated in FIG. 11). The circular or spherical shape of the cement grooves is particularly advantageous because the shape securely bonds in cement and prohibits movement of the component.

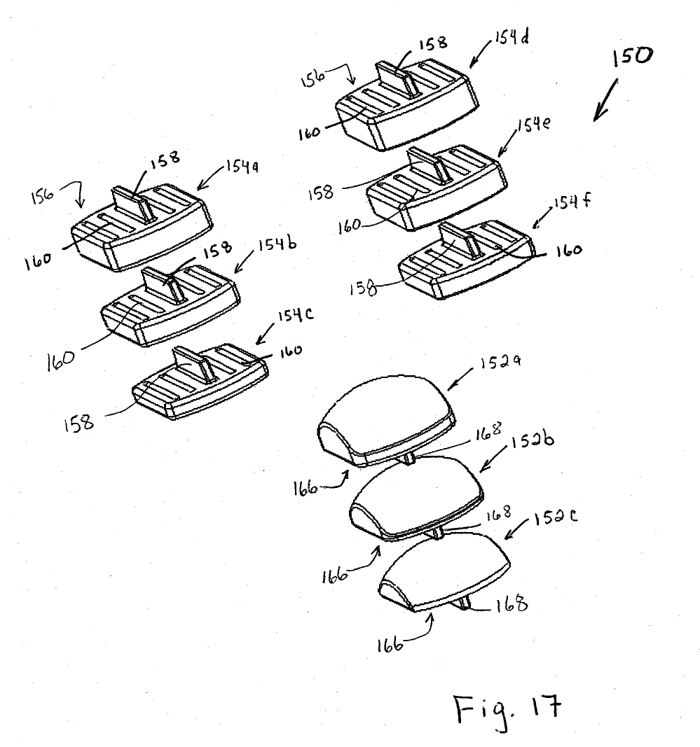

FIGS. 12-14 show a convex component 120 having a fixation surface 122 with a plurality of cement grooves 124 and a dovetail keel 126. These grooves have a dovetail shape 128 in cross section (illustrated in FIG. 14). The dovetail shape of the cement grooves is particularly advantageous because the shape securely bonds in cement and prohibits movement of the component.

FIGS. 15 and 16 further illustrate more variations and combinations of keels and cement grooves. FIG. 15 shows a convex component 130 having a fixation surface 132 with a plurality of cement grooves 134 and a dovetail keel 136. The grooves are formed in a matrix shaped as a crisscross. This crisscross has a grid shape with a plurality of grooves that are parallel. These parallel grooves are perpendicular to a plurality of other parallel grooves. These grooves intersect to form the grid or crisscross shape.

FIG. 16 shows a concave component 140 having a fixation surface 142 with a plurality of cement grooves 144 and a dovetail keel 146. The grooves are formed in a matrix shaped as a crisscross.

In both FIGS. 15 and 16, the crisscross shape of the cement grooves is particularly advantageous because the shape securely bonds in cement and prohibits movement of the component.

The ankle prostheses of the present invention can be utilized in a system comprising a plurality of differently sized concave and convex components that are interchangeable and useable to form various ankle prostheses. One skilled in the art will appreciate that this system includes the various embodiments taught in this disclosure. One such system 150 is shown in FIG. 17.

System 150 includes three differently sized convex components 152a-152c and six differently sized concave components 154a-154f The components can be generally sized to have small, medium, and large sizes. Each size can, for example, vary in thickness. More specifically, convex components 152a-152c can have sizes of neutral, +2 mm, and +4 mm. Further, concave components 154a-154f can have sizes of neutral, +2 mm, +4 mm wherein each thickness further comes either in a shallow articulation surface (−60° sphere) or in a deep articulation surface (−75° sphere).

Further, the concave components have a fixation surface 156 with both a keel 158 and cement grooves 160. The grooves and keel can have the configurations shown and discussed in connection with FIGS. 1B, 2B, and 7-16. The combination of grooves and a keel is particularly advantageous for strong cement fixation in system 150.

The convex components 152a-152c are also provided with a fixation surface 166. This fixation surface can include a keel 168 and/or cement grooves. The grooves and keel can have the configurations shown and discussed in connection with FIGS. 1B, 2B, and 7-16. The combination of grooves and a keel is particularly advantageous for strong cement fixation in system 150.

As noted, one of the advantages of the present invention is that the components can be fabricated from metal and/or polymer. These metals include Cobalt Chrome (CoCr), Titanium alloy, Commercially pure Titanium, Stainless Steel, Titanium nitride on CoCr, or Diamond-like Coating on Titanium or CoCr. Further, these polymers include many commercially available forms known in the art, such as polyethylene and UHMWPe. Examples of suitable materials are Metasul® and Durasul® articulation components manufactured by Centerpulse Orthopedics Inc. of Austin, Tex.

The figures illustrate talar and tibial components that utilize sphere-on-sphere articulating surfaces. One skilled in the art will appreciate that the fixation surfaces employing a keel and/or cement grooves of the present invention can be utilized with other types of articulating surfaces for ankle prostheses, such as those not using a spherical interface. Further, the fixation surface of the present invention can be utilized with other implantable orthopedic prostheses, such as knees, hips, and shoulders.

Although illustrative embodiments have been shown and described, a wide range of modifications, changes, and substitutions is contemplated in the foregoing disclosure; and some features of the embodiments may be employed without a corresponding use of other features. Accordingly, it is appropriate that the appended claims be construed broadly and in a manner consistent with the scope of the embodiments disclosed herein.

What is claimed is:

1) An ankle prosthesis, comprising:
a talar component having a body with a talar fixation surface and an oppositely disposed talar articulating surface, wherein the talar fixation surface is adapted to engage a natural talus, has an elongated keel projecting outwardly from the fixation surface, and has at least one cement groove; and
a tibial component having a body with a tibial fixation surface and an oppositely disposed tibial articulating surface, wherein the tibial fixation surface is adapted to engage a natural tibia, has an elongated keel projecting outwardly from the fixation surface, and has at least one cement groove.

2) The ankle prosthesis of claim 1 wherein the keels on the talar and tibial fixation surfaces have a shape selected from one of a straight rectangular shape, a dovetail shape, an I-beam shape, and a T-shape.

3) The ankle prosthesis of claim 1 wherein keels on the talar and tibial fixation surfaces have a dovetail shape.

4) The ankle prosthesis of claim 3 wherein the grooves on the talar and tibial fixation surfaces have a dovetail shape.

5) The ankle prosthesis of claim 1 wherein the keels on the talar and tibial fixation surfaces have an I-beam shape.

6) The ankle prosthesis of claim 5 wherein the grooves on the talar and tibial fixation surfaces have a circular shape in cross section.

7) The ankle prosthesis of claim 1 wherein the grooves form a crisscross matrix.

8) The ankle prosthesis of claim 7 wherein the cement grooves have a shape selected from one of circular shape and dovetail shape.

9) An ankle prosthesis, comprising:
a talar component having a body with a talar fixation surface and an oppositely disposed talar articulating surface, wherein the talar fixation surface is adapted to connect to a natural talus and has a plurality of parallel cement grooves; and
a tibial component having a body with a tibial fixation surface and an oppositely disposed tibial articulating surface, wherein the tibial fixation surface is adapted to connect to a natural tibia and has an elongated keel extending downwardly from the fixation surface.

10) The ankle prosthesis of claim 9 wherein the cement grooves have a spherical shape.

11) The ankle prosthesis of claim 10 wherein the keel has a dovetail shape.

12) The ankle prosthesis of claim 9 wherein the cement grooves are parallel and the keel has an elongated rectangular shape.

13) The ankle prosthesis of claim 9 wherein the keel has a shape selected from one of straight rectangular shape, a dovetail shape, an I-beam shape, and a T-shape.

14) The ankle prosthesis of claim 9 wherein the cement grooves have a shape selected from one of circular shape, dovetail shape, and crisscross shape.

15) The ankle prosthesis of claim 9 wherein the talar and tibial components have a substantially rectangular shape in cross section.

16) The ankle prosthesis of claim 15 wherein the talar and tibial components have rounded ends.

17) A prosthetic ankle system, comprising:
a plurality of differently sized talar components, wherein each talar component has a talar fixation surface adapted to engage a natural talus and a talar articulating surface; and
a plurality of differently sized tibial components, wherein each tibial component has a tibial fixation surface adapted to engage a natural tibia and a tibial articulating surface adapted to engage the talar articulating surface, wherein at least one talar component and at least one tibial component together form an ankle prosthesis adapted for use at an ankle joint, and wherein the talar and tibial fixation surfaces include an elongated keel and at least one cement groove.

18) The prosthetic ankle system of claim 17 wherein the cement grooves have a shape selected from one of circular shape, dovetail shape, and crisscross shape.

19) The prosthetic ankle system of claim 18 wherein each talar component has a different thickness and each tibial component has a different thickness.

20) The prosthetic ankle system of claim 19 wherein the keels have a shape selected from one of a straight rectangular shape, a dovetail shape, an I-beam shape, and a T-shape.
